(19) United States
(12) Patent Application Publication
(10) Pub. No.: US 2011/0206928 A1
(43) Pub. Date: Aug. 25, 2011

(54) Reinforced Fibers and Related Processes

(76) Inventors: Jeffrey P. Maranchi, Clarksburg, MD (US); Robert C. Matteson, III, Ellicott City, MD (US); Michael Rooney, Baltimore, MD (US); Paul D. Wienhold, Baltimore, MD (US)

(21) Appl. No.: 12/862,003

(22) Filed: Aug. 24, 2010

Related U.S. Application Data
(60) Provisional application No. 61/236,358, filed on Aug. 24, 2009.

(51) Int. Cl. D02G 3/02; D02G 3/36; C12P 19/04

(52) U.S. Cl. 428/365; 435/101

(57) ABSTRACT

A reinforced fiber is disclosed including a base material composed of a reinforcement ply having interstitial spaces; and a bacterial cellulose interwoven over the reinforcement ply and throughout the interstitial spaces. The reinforced fiber is obtained by (a) providing a base material composed of a reinforcement ply having interstitial spaces; and (b) contacting the base material with an effective bacteria in a microbial fermentation synthesis process for a time period sufficient to grow bacterial cellulose throughout the interstitial spaces of the reinforcement ply thereby providing a reinforced fiber interwoven with the bacterial cellulose. Also disclosed reinforced silicon carbide-containing nanofibers and processes for their preparation.

### CROSS-REFERENCE TO RELATED APPLICATIONS

This application claims the benefit of prior filed co-pending U.S. Provisional to Maranchi et al., Application No. 61/236,358, filed Aug. 24, 2009, the contents of which are incorporated by reference herein.

### BACKGROUND OF THE INVENTION

1. Field of the Invention

The present invention generally relates to reinforced fibers derived from bacterial cellulose, method of producing the same and products produced therefrom.

2. Description of the Related Art

The discovery of nanomaterials and particularly, those formed from carbon, has been of great interest to many researchers. This interest has led to various processes and applications being developed to exploit the unique properties of these materials. Of the many potential areas of application, the area of perhaps the greatest interest is the development of engineered composite materials using nanotubes or other nanostructures and devices. Examples of contemplated products using these materials include for example, as black body emitters (e.g., thermophotovoltaic devices), electronic applications (e.g., high surface area, high temperature sensing applications), and near indestructible armor.

Unfortunately, composite materials and specifically, methods utilizing nanotubes or other nanostructures to improve the properties of materials by forming nano-composite matrices, particularly those based upon glass, ceramic, or metal; have met various challenges and shortcomings. These shortcomings include poor dispersion of the nanotubes in the matrix material, primarily due to Van der Waals' forces; poor alignment and orientation of the nanotubes in the matrix; short lengths of the nanotubes relative to defect sizes in the composite matrices; and difficulties associated with handling randomly oriented nanotubes in an industrial scale process.

Prior to the current interest in nanomaterials and their application, artisans devised various strategies for improving the physical properties of materials by forming composite materials. One such approach to increasing the strength of a glass or ceramic is to incorporate relatively large fibers or fiber bundles into the glass or ceramic material. Typically, such fibers are comprised of carbon or silicon carbide.

Microbial cellulose produced by certain microorganisms has been known and studied for over a hundred years. Microbially derived cellulose possesses distinct characteristics not found in plant cellulose, including high water content similar to hydrogels and exceptional strength like PTFE. Microbial cellulose can be synthesized in various shapes or sizes, and has excellent shape retention. These properties are mostly attributed to its unique laminar microfibrillar three-dimensional structure. The microfibrils arranged in a nonwoven manner are about 200 times finer than plant cellulose such as cotton fibers, yielding tremendous surface area per unit volume.

Even with the multitude of properties, microbial cellulose has not been fully utilized, and thus, limited applications have been suggested. For example, the use of microbially derived cellulose in the medical industry has been limited to liquid loaded pads, wound dressings and other topical applications.

There is a continued need for improved reinforced fibers that have suitable properties, which can be made in a simple, cost efficient manner.

### SUMMARY OF THE INVENTION

In accordance with one embodiment of the present invention, there is provided a reinforced fiber comprising a base material composed of a reinforcement ply having interstitial spaces; and a bacterial cellulose interwoven over the reinforcement ply and throughout the interstitial spaces.

In accordance with a second embodiment of the present invention, there is provided a process for preparing a reinforced fiber comprising (a) providing a base material composed of a reinforcement ply having interstitial spaces; and (b) contacting the base material with an effective bacteria in a microbial fermentation synthesis process for a time period sufficient to grow bacterial cellulose throughout the interstitial spaces of the reinforcement ply thereby providing a reinforced fiber interwoven with the bacterial cellulose.

In accordance with a third embodiment of the present invention, there is provided a method for making a reinforced silicon carbide-containing nanofiber which comprises (a) providing nanofibers derived from pellicles of bacteria cellulose; (b) forming a silicon oxide-containing on the surface of the nanofibers with a silicon oxide forming coating solution; and (c) heating the silicon oxide-containing coated nanofibers in the presence of a non-oxygen-containing fluid to a temperature and for time sufficient to form reinforced silicon carbide-containing nanofibers.

In accordance with a fourth embodiment of the present invention, there is provided a reinforced silicon carbide-containing nanofiber obtained from a process comprising (a) providing nanofibers derived from pellicles of bacteria cellulose; (b) forming a silicon oxide-containing coating on the surface of the nanofibers with a silicon oxide forming coating solution; and (c) heating the silicon oxide-containing coated nanofibers in the presence of a non-oxygen-containing fluid (e.g., flowing argon gas) to a temperature and for a time period sufficient to form reinforced silicon carbide-containing nanofibers.

In accordance with a fifth embodiment of the present invention, there is provided a composite comprising one or more reinforced silicon carbide-containing nanofibers obtained from a process comprising (a) providing nanofibers derived from pellicles of bacteria cellulose; (b) forming a silicon oxide-containing on the surface of the nanofibers with a silicon oxide forming coating solution; and (c) heating the silicon oxide-containing coated nanofibers in the presence of a non-oxygen-containing fluid to a temperature and for a time period sufficient to form reinforced silicon carbide-containing nanofibers.

In accordance with a sixth embodiment of the present invention, there is provided a product comprising one or more reinforced silicon carbide-containing nanofibers obtained from a process comprising (a) providing nanofibers derived from pellicles of bacteria cellulose; (b) forming a silicon oxide-containing on the surface of the nanofibers with a silicon oxide forming coating solution; and (c) heating the silicon oxide-containing coated nanofibers in the presence of a non-oxygen-containing fluid to a temperature and for time period sufficient to form reinforced silicon carbide-containing nanofibers.

### BRIEF DESCRIPTION OF THE DRAWINGS

FIG. 1 is a flow chart illustrating one embodiment for preparing reinforced fibers from a base material composed of a reinforcement ply interwoven with bacterial cellulose.

FIG. 2 is a cross-sectional view of an example of a reinforced fiber including a base material composed of a plurality of fiber bundles of fiberglass defining interstitial spaces between the adjacent fiber bundles and a bacterial cellulose interwoven over the fiber bundles and throughout the interstitial spaces.

FIG. 3 is a cross-sectional view of an example of a reinforced fiber including a base material composed of a plurality of layers each including a plurality of fiber bundles of fiberglass defining interstitial spaces between the adjacent fiber bundles and a bacterial cellulose interwoven over the plurality of layers and throughout the interstitial spaces.

FIG. 4 is a flow chart illustrating one embodiment for preparing reinforced silicon carbide-containing nanofibers.

### DETAILED DESCRIPTION OF PREFERRED EMBODIMENT(S)

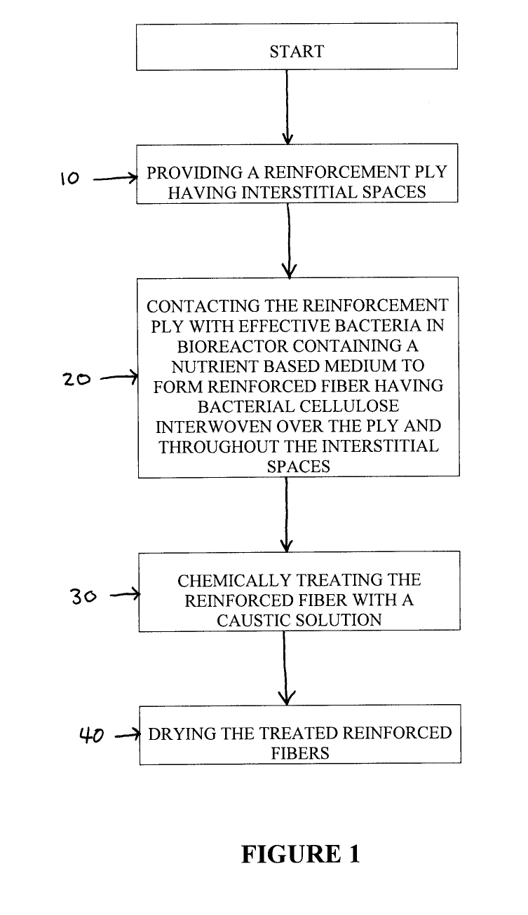

One aspect of the present invention is directed to a reinforced fiber comprising a base material composed of a reinforcement ply having interstitial spaces and a bacterial cellulose interwoven over the reinforcement ply and throughout the interstitial spaces. Referring now to FIG. 1, there is shown a flow chart illustrating a process for preparing the reinforced fibers of one embodiment of the present invention. The initial step 10 of the process is providing a base material composed of a reinforcement ply having interstitial spaces. The reinforcement ply includes any woven or nonwoven material having sufficient interstitial space capable of receiving the grown bacteria and form reinforced fibers interwoven with the bacterial cellulose. Representative examples of reinforcement plies for use herein include woven and/or non-woven carbon fibers, fiberglass, Kevlar, plastic, cloth, nylon and the like.

In one embodiment, a base material is composed of reinforcement ply including a plurality of fiber bundles of fiberglass defining interstitial spaces between the adjacent fiber bundles.

In another embodiment, a base material is composed of a plurality of layers of reinforcement plies each including a plurality of fiber bundles of fiberglass defining an interstitial space between the adjacent fiber bundles.

The next step 20 is contacting the base material with an effective bacteria in a microbial fermentation synthesis process for a time period sufficient to grow bacterial cellulose from the bottom side of the base material to the top side of the base material and throughout the interstitial spaces thereby providing a reinforced fiber interwoven with the bacterial cellulose.

Effective bacteria that can produce such bacterial cellulose include, by way of example, Acetobacter pasteurianus ATCC 23769, FERM BP-4176, Acetobacter aceti, Acetobacter xylinum, Acetobacter rancens, Sarcina ventriculi, Bacterium xyloides and bacteria belonging to the genus Pseudomonas, the genus Agrobacterium, the genus Rhizobium, etc. In one embodiment, the microorganisms are Acetobacter xylinum.

The base material is contacted with the effective bacteria in a bioreactor containing a nutrient based medium. The nutrient based medium contains a carbon source, a nitrogen source, inorganic salts and, if necessary, organic minor nutrients such as amino acids, vitamins, etc. A suitable carbon source includes glucose, sucrose, maltose, starch hydrolysate, molasses, etc., but ethanol, acetic acid, nitric acid, etc., may also be used singly or in combination with the above-described sugars. A suitable nitrogen source includes organic or inorganic nitrogen sources such as ammonium salts, e.g. ammonium sulfate, ammonium chloride, ammonium phosphate, etc., nitrates, urea, peptone and the like. Inorganic salts are minor phophates, magnesium salts, calcium salts, iron salts, manganese salts, etc. As organic nutrients amino acids, vitamins, fatty acids, nucleic acids, etc. are used. Furthermore, peptone, casamino acid, yeast extracts, soybean protein hydrolysates, etc., containing these nutrients may be used. When using auxotrophs requiring amino acids, etc., for growth, it is necessary to add required nutrients.

Suitable bioreactors are selected which minimize evaporation and provide adequate oxygen-limiting conditions. Oxygen-limiting conditions may be varied depending upon the desired water content and thickness of the cellulose film. Generally, under oxygen limited conditions, oxygen is present in an amount of 10% to 20% of the total gas present at the air liquid interface. The bioreactor can be composed of a plastic box fitted with an airtight cover or a limited gas-permeable cover. Dimensions of the bioreactor can vary in configuration depending on the shape and size of the cellulose pellicles being produced. By limiting the amount of oxygen in the medium, it is hypothesized that the bacteria such as Acetobacter xylinum utilizes the carbon available in the medium to produce more cellulose instead of using it for reproduction, thereby increasing the total yield of cellulose.

The cultivation method is not particularly limited, and may be static culture, agitation culture (aeration agitation culture, shaking culture, oscillation culture, air lift type culture) or the like.

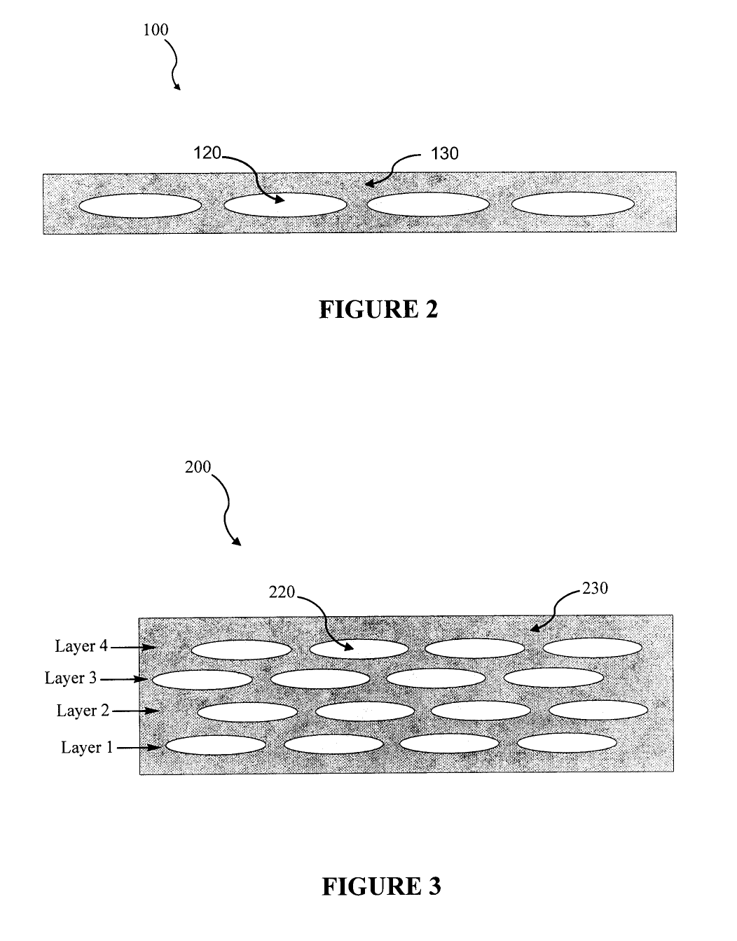

Generally, the bioreactor is sealed and incubated in a controlled environment until growth of the bacterial cellulose throughout the interstitial spaces in the reinforcement ply is complete. In other words, the growth of the bacterial cellulose is allowed to continue from the bottom side of the ply to the top side of the ply and throughout the interstitial spaces. As generally depicted in FIG. 2, in the case of reinforced fiber 100 including a base material composed of reinforcement ply including a plurality of fiber bundles 120 of fiberglass defining interstitial spaces between the adjacent fiber bundles, the bacterial cellulose 130 is allowed to grow such that it is interwoven over the fiber bundles 120 and throughout the interstitial spaces.

Further, as generally depicted in FIG. 3, in the case of a reinforced fiber 200 including a base material composed of a plurality of layers 1-4 of reinforcement plies, each layer including a including a plurality of fiber bundles 220 of fiberglass defining interstitial spaces between the adjacent fiber bundles, the bacterial cellulose 230 is allowed to grow such that it is interwoven over the plurality of layers, for example four layers are described herein, but the layers are not limited to four, and throughout the interstitial spaces.

The culture conditions may be conventional: for example, at a pH of about 3 to about 9 and at a temperature of about 1 to about 40° C. The time period for growth of the bacterial cellulose will range from about 1 to about 100 days. Depending on the desired thickness, which corresponds to certain cellulose content per unit area, the fermentation is stopped and the interwoven fiber is removed from the bioreactor. The excess medium contained in the fibers is then removed by standard separation techniques such as compression or centrifugation prior to chemical cleaning and subsequent processing of the pellicle.

Following production of the interwoven fibers, the next step 30 involves removing the interwoven fibers from the bioreactor and chemically treating them to remove bacterial by-products and residual media. Typically, the interwoven fibers are subjected to one or more chemical washing steps using a caustic solution to remove viable organisms and pyrogens (endotoxins) produced by bacteria from the pellicle. A caustic solution can contain an alkali metal hydroxide such as sodium hydroxide or an alkali metal carbonate such as potassium or sodium carbonate at concentrations of about 0.2 M to about 2 M. The time involved in this process can be as much as 4 hours at a temperature of about 40° C. to about 85° C. The treated interwoven fibers may then be rinsed with filtered water to reduce microbial contamination (bioburden). If desired, the refractive index of the fiber can be adjusted during this step by treating the cellulose with a suitable chemical such as, for example, acetic anhydride based solutions which can decrease the refractive index by replacing hydroxyl groups with acetyl groups on the cellulose chains.

After the interwoven fibers have been chemically treated, the next step 40 involves drying the treated fibers. Drying can be carried out by any technique known in the art but within the temperature range wherein bacterial cellulose is not decomposed. For example, drying can be carried out by supercritical drying, freeze-drying, ambient pressure drying, heating, chemical drying, e.g., HMDS drying, and the like. In one embodiment, drying is carried out by supercritical drying. This is where the liquid within the coated nanofibers is removed by, for example, prior solvent exchange with CO₂ followed by supercritical drying. The drying process should remove greater than 99% of all water within the cellulose.

If desired, any remaining unwanted residue remaining on the surface of the resulting fiber can be removed by techniques known in the art, e.g., a doctor blade, uv, etc.

While not wishing to be bound by any particular theory, it is believed that the reinforced fibers of this embodiment advantageously allow for bacterial cellulose to grow in the interstitial space in the reinforcement ply, e.g., the interstitial space between adjacent fibers such as in the case of individual reinforcement fibers, the interstitial space between fiber bundles, or the interstitial space between fibers in adjacent plies in the through thickness direction when laying up multiple plies, such that a 3-D interconnectivity may be achieved and hence improved mechanical reinforcement properties in directions not normally reinforced in conventionally processed composite materials. For example, in the case of a conventional multi-ply layup of glass fiber woven fabric, there are no dedicated reinforcement elements in the through thickness direction of the layup. By modifying the conventional process and infiltrating a 3-D network of bacterial cellulose nanofibers interstitially through the spaces between, for example, fiber bundles within one ply, and vertically through the thickness of the multi-ply layup, it is believed that the mechanical properties (e.g., stiffness and ultimate tensile strength) of the resultant reinforced fiber in the through-thickness direction will be positively impacted and additional reinforcement effect in the in-plane directions.

The reinforced interwoven fibers can be useful in a variety of different products. For example, the reinforced interwoven fibers can be used as reinforcements in a composite material. The composite can be formed by, for example, injection molding, hot pressing and the like, as known in the art. Alternatively, the reinforced interwoven fibers can be used as a reinforcing layer in making, for example, fishing poles. Other products include composites for making, by way of example, windmill blades, aerospace structures, and boat/maritime structures. For example, the reinforced fibers can be used in forming a heat shield for space vehicles.

In one embodiment, fiber reinforced composite structures are formed wherein the reinforced fibers of the present invention are the predominant load bearing members and a resinous matrix material function primarily to hold the fibers together.

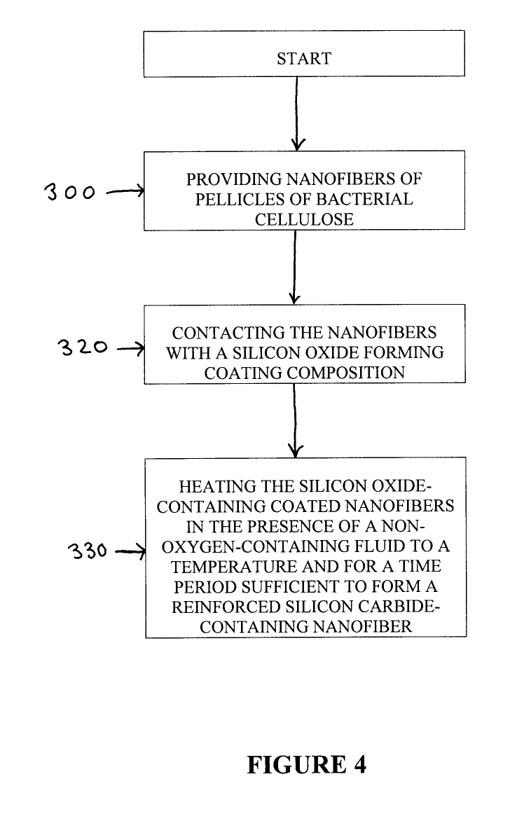

In accordance with another aspect of the present invention, an embodiment is directed to reinforced silicon carbide-containing nanofibers. Referring now to FIG. 4, there is shown a flow chart illustrating a process for preparing a reinforced silicon carbide-containing nanofiber. The initial step 300 of the process is providing nanofibers derived from pellicles of bacterial cellulose. In general, the step of providing nanofibers derived from pellicles of bacterial cellulose involves first forming pellicles of bacterial cellulose. The step of forming pellicles of bacterial cellulose is well known in the art. Microorganisms that produce such bacterial cellulose include, by way of example, Acetobacter pasteurianus ATCC 23769, FERM BP-4176,Acetobacter aceti, Acetobacter xylinum, Acetobacter rancens, Sarcina ventriculi, Bacterium xyloides and bacteria belonging to the genus Pseudomonas, the genus Agrobacterium, the genus Rhizobium, etc. In one embodiment, the microorganisms are Acetobacter xylinum.

In forming pellicles of bacterial cellulose, microorganisms such as Acetobacter xylinum are cultured in a bioreactor containing a nutrient based medium. The nutrient based medium contains a carbon source, a nitrogen source, inorganic salts and, if necessary, organic minor nutrients such as amino acids, vitamins, etc. A suitable carbon source includes glucose, sucrose, maltose, starch hydrolysate, molasses, etc., but ethanol, acetic acid, nitric acid, etc., may also be used singly or in combination with the above-described sugars. A suitable nitrogen source includes organic or inorganic nitrogen sources such as ammonium salts, e.g. ammonium sulfate, ammonium chloride, ammonium phosphate, etc., nitrates, urea, peptone and the like. Inorganic salts are minor phophates, magnesium salts, calcium salts, iron salts, manganese salts, etc. As organic nutrients amino acids, vitamins, fatty acids, nucleic acids, etc. are used. Furthermore, peptone, casamino acid, yeast extracts, soybean protein hydrolysates, etc., containing these nutrients may be used. When using auxotrophs requiring amino acids, etc., for growth, it is necessary to add required nutrients.

Suitable bioreactors are selected which minimize evaporation and provide adequate oxygen-limiting conditions. Oxygen-limiting conditions may be varied depending upon the desired water content and thickness of the cellulose film. Generally, under oxygen limited conditions, oxygen is present in an amount of 10% to 20% of the total gas present at the air liquid interface. The bioreactor can be composed of a plastic box fitted with an airtight cover or a limited gas-permeable cover. Dimensions of the bioreactor can vary in configuration depending on the shape and size of the cellulose pellicles being produced. By limiting the amount of oxygen in the medium, it is hypothesized that the bacteria such as Acetobacter xylinum utilizes the carbon available in the medium to produce more cellulose instead of using it for reproduction, thereby increasing the total yield of cellulose.

The cultivation method is not particularly limited, and may be static culture, agitation culture (aeration agitation culture, shaking culture, oscillation culture, air lift type culture) or the like.

Generally, the bioreactor trays are sealed and incubated in a controlled environment until growth of a pellicle of the bacterial cellulose is complete. The culture conditions may be conventional: for example, at a pH of about 3 to about 9 and at a temperature of about 1 to about 40° C., with the culture being performed for about 1 to about 100 days. Depending on the desired thickness, which corresponds to certain cellulose content per unit area, the fermentation is stopped and the pellicle is removed from the bioreactor. The excess medium contained in the pellicle is then removed by standard separation techniques such as compression or centrifugation prior to chemical cleaning and subsequent processing of the pellicle.

Following production of the cellulose pellicles, the pellicles are removed from the bioreactor trays and are chemically treated to remove bacterial by-products and residual media. Typically, the cellulose pellicles are subjected to one or more chemical washing steps using a caustic solution to remove viable organisms and pyrogens (endotoxins) produced by bacteria from the pellicle. A caustic solution can contain an alkali metal hydroxide such as sodium hydroxide or an alkali metal carbonate such as potassium or sodium carbonate at concentrations of about 0.2 M to about 2 M. The time involved in this process can be as much as 4 hours at a temperature of about 40° C. to about 85° C. The treated pellicles may then be rinsed with filtered water to reduce microbial contamination (bioburden).

If desired, the chemically processed cellulose films (pellicles) can be exposed to a “bleaching” process to attain a “whitening” effect on the material. A typical bleaching solution of hydrogen peroxide is in the range of about 0.25% to about 3% and is prepared from concentrated hydrogen peroxide and filtered water.

In a controlled environment, the pellicles are compressed to the desired thickness and to remove any excess water present in the pellicle. For example, the washed pellicles can be placed between two glass plated with a suitable water absorbing medium, e.g., a paper towel, and then pressure is applied to remove any excess water. It is the thickness of the compressed film that achieves the final desired density of the microbially-derived cellulose fiber. Thus the original fill volume as well as the compression steps are integral to attain the desired density that affects the strength, integrity, and function of the cellulose. The films can be cut to various shapes and sizes that those skilled in the art will understand.

The next step 320 involves contacting the nanofibers of the cellulose pellicles with a silicon oxide forming coating composition to form a silicon oxide-containing coating on the surface thereof. A silicon oxide forming coating composition can contain one or more silicon alkoxides in an alcohol and an aqueous medium. Useful silicon alkoxides include those represented by the general formula (R)ₙSi(OR₄)₄₋ₙ, wherein each of R and R₁, independently, is H or C₁ to C₄ alkyl, and n is an integer from 0 to 3. The most commonly used silicon alkoxide is tetraethylorthosilicate (TEOS). Typically the alcohols used are methanol, ethanol, and the like and a mixture thereof. The aqueous media can be de-ionized water or distilled water. Generally, the amount of silicon alkoxide present in the solution can range from about 0.01 vol. % to about 20 vol. %. The amount of alcohol present in the solution can range from about 20 vol. % to about 99.99 vol. %.

The silicon oxide forming coating composition can be applied to the nanofibers of the cellulose pellicles by known processes to form coated nanofibers. For example, the wet nanofibers can be dried and then contacted with the silicon oxide forming coating composition by, for example, any spraying or by dipping process, to form a silicon oxide coating on the surface thereof. The wet nanofibers can be dried at a temperature of about 60° C. to about 180° C. for a time period of about 1 hour to about 12 hours. In another embodiment, the coating can be applied to the nanofibers by first extracting the washing solution from the wet nanofibers and then treating the extracted wet nanofibers with a sufficient amount of the coating composition to form a silicon oxide coating on the surface thereof. A sufficient amount of the coating composition can be from about 1% to about 100% of the equivalent weight of the dried cellulose being treated.

After the silicon oxide-containing coating has been formed on the surface of the nanofibers, the coated nanofibers are dried. Drying can be carried out by any technique known in the art but within the temperature range wherein bacterial cellulose is not decomposed. For example, drying can be carried out by supercritical drying, freeze-drying, ambient pressure drying, heating, chemical drying, e.g., HMDS drying, and the like. In one embodiment, drying is carried out by supercritical drying. This is where the liquid within the coated nanofibers is removed by, for example, prior solvent exchange with CO₂ followed by supercritical drying to remove substantially all remaining water, ethanol and excess alkoxide.

The next step 330 involves heating the silicon oxide-containing coated nanofibers in the presence of a non-oxygen-containing fluid to a temperature and for a time period sufficient to form reinforced silicon carbide-containing nanofibers. In one embodiment, the silicon oxide-containing coated nanofibers are heated such that the nanofibers can undergo as complete a carbothermal reduction. Suitable non-oxygen-containing fluids include, by way of example, argon, nitrogen, and/or helium gases. The temperature for heating the nanofibers is at least about 1400° C. In one embodiment, the temperature for heating the nanofibers can range from about 1400° C. to about 1700° C. A sufficient time period can range from about 4 hours to about 48 hours.

The resulting reinforced silicon carbide-containing nanofibers are monolithic. In addition, the resulting reinforced silicon carbide-containing nanofibers can be of any size or shape.

The reinforced silicon carbide-containing nanofibers can be useful in a variety of different products. For example, the reinforced silicon carbide-containing nanofibers can be used as reinforcements in a composite material such as a high temperature composite material in, for example, engines or electronic applications. In one embodiment, the reinforced silicon carbide-containing nanofibers can be used in forming an abrasive material. In one embodiment, the reinforced silicon carbide-containing nanofibers can be used in forming a brake pad. In one embodiment, the reinforced silicon carbide-containing nanofibers can be used as black body emitters for thermophotovoltaic devices. In one embodiment, the reinforced silicon carbide-containing nanofibers can be used as layer in forming, for example, a personal protective device such as a bullet proof vest. Methods for making the foregoing products are within the purview of one skilled in the art.

In another embodiment, the reinforced silicon carbide-containing nanofibers can be used as a composite reinforcement in an optically clear, ultra-high strength visually transparent composite. In order to achieve this, a partial oxidation step is carried out to partially oxidize the surface of the silicon carbide-containing nanofibers to effectively grade the refractive index from greater than about 2 down to refractive index in the 1.6 range. The partial oxidation step is carried out in a low oxidizing fluid such as a gas, e.g., about 0.1% to about 1% oxygen/balance argon or nitrogen, at an elevated temperature of about 400° C. to about 1000° C. for about 0.1 hour to about 48 hours.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

What is claimed is:

1. A reinforced fiber comprising a base material composed of a reinforcement ply having interstitial spaces; and a bacterial cellulose interwoven over the reinforcement ply and throughout the interstitial spaces.

2. The reinforced fiber of claim 1, wherein the reinforcement ply is a woven or nonwoven material selected from the group consisting of fiberglass, plastic, Kevlar, cloth, carbon, and ceramic.

3. The reinforced fiber of claim 1, comprising a base material composed of a reinforcement ply including a plurality of fiber bundles of fiberglass defining interstitial spaces between the adjacent fiber bundles and a bacterial cellulose interwoven over the fiber bundles and throughout the interstitial spaces.

4. The reinforced fiber of claim 1, comprising a base material composed of a plurality of layers of reinforcement plies, each layer of reinforcement ply including a plurality of fiber bundles of fiberglass defining interstitial spaces between the adjacent fiber bundles, and a bacterial cellulose interwoven over the plurality of layers and throughout the interstitial spaces.

5. The reinforced fiber of claim 1, wherein the bacterial cellulose is derived from bacteria selected from the group consisting of Acetobacter pasteurianus ATCC 23769, FERM BP-4176, Acetobacter aceti, Acetobacter xylinum, Acetobacter rancens, Sarcina ventriculi, Bacterium xyloides and bacteria belonging to the genus Pseudomonas, the genus Agrobacterium, and the genus Rhizobium.

6. A process comprising (a) providing a base material composed of a reinforcement ply having interstitial spaces; and (b) contacting the base material with an effective bacteria in a microbial fermentation synthesis process for a time period sufficient to grow bacterial cellulose throughout the interstitial spaces of the reinforcement ply thereby providing a reinforced fiber interwoven with the bacterial cellulose.

7. The process of claim 6, wherein the reinforcement ply is a woven or nonwoven material selected from the group consisting of fiberglass, plastic, Kevlar, cloth, carbon and ceramic.

8. The process of claim 6, wherein the base material is composed of a reinforcement ply including a plurality of fiber bundles of fiberglass defining interstitial spaces between the adjacent fiber bundles and the bacterial cellulose is interwoven over the fiber bundles and throughout the interstitial spaces.

9. The process of claim 6, wherein the base material is composed of a plurality of layers of reinforcement plies, each layer of reinforcement ply including a plurality of fiber bundles of fiberglass defining interstitial spaces between the adjacent fiber bundles, and the bacterial cellulose is interwoven over the plurality of layers and throughout the interstitial spaces.

10. The process of claim 8, wherein the effective bacteria is Acetobacter xylinum.

11. The process of claim 6, wherein the reinforcement ply is contacted with the effective bacteria for a time period of about 1 day to about 100 days.

12. The process of claim 6, further comprising treating the resulting reinforced fiber interwoven with the bacterial cellulose with a caustic solution

13. The process of claim 12, wherein the caustic solution comprises an alkali metal hydroxide or carbonate.

14. The process of claim 12, wherein the caustic solution comprises sodium hydroxide.

15. The process of claim 6, further comprising drying the resulting reinforced fiber interwoven with the bacterial cellulose.

16. The process of claim 15, wherein the step of drying comprises supercritical drying, freeze-drying, ambient pressure drying, heating, or chemical drying the resulting reinforced fiber.

17. The process of claim 6, further comprising
treating the resulting reinforced fiber interwoven with the bacterial cellulose with a caustic solution; and
drying the treated reinforced fiber.

18. The process of claim 6, further comprising injection molding, hot pressing, vacuum assisted molding, or UV curing the resulting reinforced fiber interwoven with the bacterial cellulose to form a molded product.

19. The process of claim 17, further comprising injection molding or hot pressing the resulting reinforced fiber interwoven with the bacterial cellulose to form a molded product.

20. A reinforced fiber obtained by the process of claim 6.
